(19) United States
(12) Patent Application Publication
(10) Pub. No.: US 2008/0208213 A1
(43) Pub. Date: Aug. 28, 2008

(54) Devices and methods for the controlled formation and closure of vascular openings

(76) Inventors: Elad Benjamin, Stanford, CA (US); Michael Elliot Gober, Palo Alto, CA (US); Carla Peinknagura, Menlo Park, CA (US); Bauback Safa, Palo Alto, CA (US); Jonathan Moulton Thomas, Nantucket, MA (US)

Correspondence Address: Bozicevic, Field & Francis LLP;Stanford University Office of Technology Licensing, 1900 University Avenue, Suite 200, East Palo Alto, CA, 94303, US

(21) Appl. No.: 11/440,698

(22) Filed: May 24, 2006

Related U.S. Application Data
(60) Provisional application No. 60/685,590, filed on May 31, 2005.

(51) Int. Cl. A61B 17/10; A61B 17/08

(52) U.S. Cl. 606/139; 606/153

(57) ABSTRACT

The present invention includes systems, devices and methods for percutaneously forming an aperture within a tissue structure or vessel and closing the aperture in a manner which optimizes hemostasis and healing. The invention in one aspect includes implantable devices which are used to seal the tissue aperture upon closure of the aperture after a percutaneous or endovascular procedure.

### CROSS REFERENCE

This filing claims the benefit of provisional patent application Ser. No. ______, entitled “Device for Controlled Opening of Vessels” filed May 25, 2005, the entirety of which is incorporated by reference.

### FIELD OF THE INVENTION

The present invention relates to the percutaneous formation and closure of vascular openings. The present invention is particularly advantageous for forming and closing large-diameter vascular openings.

### BACKGROUND OF THE INVENTION

Access to patient blood vessels is necessary for a wide variety of diagnostic and therapeutic purposes. For example, intravascular catheters are introduced to both the arterial vasculature and the venous vasculature, typically using either surgical cut down techniques or percutaneous introduction techniques in which an opening is created in the wall of a vessel situated relatively close to the skin surface.

The continued popularization of minimally invasive and endovascular procedures and the advent of devices and instrumentation for performing such procedures has seen a concurrent proliferation in the development of vessel closure devices for percutaneous procedures. These devices include clips, staples, automated suturing mechanisms, biologic plugs, fillers, glues and the like. These devices have the advantage of reducing costs and decreasing the length of hospitalizations as well as obviating the need for prolonged manual or mechanical pressure at the wound site. However, while these devices have revolutionized vascular closure in percutaneous surgery, they are designed for sealing exclusively small arteriotomy openings (6-8 F).

With the introduction of a greater number and variety of intravascular techniques, including angioplasty, atherectomy, endovascular aneurysm repair, minimally invasive cardiac surgery, and the like, a need has arisen to provide relatively large diameter access to the vasculature. Thus, access sheaths having a diameter of 16 F or greater are now commonly used.

While some surgeons have used existing vascular closure devices to close large arteriotomy sites, such has proven difficult, unreliable, and therefore not widely-adopted. Without the availability of closure devices for larger vascular access sites, open approaches continue to be used with larger skin and vessel incisions in order to achieve proper apposition of the vessel walls and adequate hemostasis upon vessel closure. Not only is there a lack of effective percutaneous devices and methodologies for the closure of large diameter vessel openings, the same can be said for the creation of such large openings.

While a wide variety of variations exist, the most commonly employed vascular access procedure is the Seldinger technique. Initial access within a target vessel is made with a needle. A guidewire is then passed through the needle into the vessel, and the needle withdrawn over the guidewire. A dilator is then passed over a guidewire to enlarge the diameter of the tissue tract so that it can accommodate a larger introducer sheath. Once the introducer sheath is in place, access to the vessel can be reliably obtained through a lumen of the sheath. Depending on the necessary size of the access opening, dilators of various sizes may be used to stretch the opening.

While nominally traumatic when used to create smaller vessel openings, larger dilators can significantly traumatize the skin and the vessel tissue. In particular, advancement of a conventional dilator through a tissue tract exerts significant axial forces on the tissue. This potentially causes injury and delamination of tissue layers in the wound tract. Furthermore, the stretching and tearing of the vessel wall results in an opening which is not uniform with an unpredictable shape and size. Moreover, the edges of the vessel opening can become friable and misshapen, making subsequent closure that much more difficult. Specifically, without the ability to provide a clean edge-to-edge alignment when closing a vessel opening, hemostasis is made difficult and endothelial and intimal growth between the vessel edges is impaired, thereby negatively affecting the wound's ability to heal properly.

Accordingly, it is desirable to provide improved vascular access formation and closure techniques for large (as well as small) diameter vascular openings, typically having diameters as large as 6 mm, preferably as large as 8 mm, or larger. It would be further advantageous to provide tools and methodologies for both the creation and closure of vascular openings whereby more predictable openings can be formed lending themselves to quicker and more effective closure. The ability to easily, quickly and successfully form and close large arteriotomy sites by percutaneous means would eliminate trauma and the resulting risks to the patient, thereby eliminating the need for performing an open procedure in the operating room, and provide for faster healing and a quicker recovery, reducing cost to the healthcare system.

### SUMMARY OF THE INVENTION

The present invention includes systems, devices and methods for percutaneously forming an aperture within a tissue structure or vessel and closing the aperture in a manner which optimizes hemostasis and healing. The invention allows for formation and closure of such vascular openings of a wide range of sizes, and is particularly useful for relatively large vascular openings having an incision length or diameter greater than about 3 mm, and particularly within the range from about 5 mm to about 8 mm in which cannulas, sheaths and other percutaneous instrumentation having sizes in the range from about 16 F to about 24 F are used. However, these vessel aperture and instrument sizes are not intended to be limiting to the invention as the invention may be configured to form/seal apertures that are smaller or larger than those stated. In certain applications, the size of the incision formed is sufficient to sealably accommodate an endovascular tool (e.g., catheter) while not being so tight as to result in stretching or dilation of the formed opening. Examples of applications in which the present invention is suitable for use include arteriotomies in the femoral arteries and veins for cardiovascular procedures such as aneurism repairs and heart valve replacements.

The invention in one aspect includes implantable devices which are used to facilitate the creation of tissue incisions having edges which maintain their shape and profile to optimally appose each other upon removal of instrumentation or the like after a percutaneous or endovascular procedure. Such devices include sutures, staples, clips, jaws, frames and the like. In certain embodiments, the implantable devices facilitate the creation of tissue incisions which are biased to a closed or sealed state, such that the incision is self-closing or sealing upon removal of instrumentation or the like after a percutaneous or endovascular procedure.

Certain variations of these implantable devices are configured to be implanted, fixed or placed subsequent to completion of the diagnostic or therapeutic procedure while others are configured to be implanted, fixed or placed prior to performing the procedure. Of the latter variety, certain of these devices are placed and affixed to the target vessel or tissue even prior to forming an incision within the vessel or tissue. Still yet, certain embodiments of the pre-incision implants allow for the precise formation of an incision which forms the access aperture and subsequent control of the opening (for the passage of instruments and other devices there through) and closing (after the removal of all instrumentation) of the access aperture. More particularly, the pre-incision implants precisely define the location, shape, size and length of the aperture to be formed, allow for controlled formation and maintenance of that aperture, and allow for precise apposition of the edges of the vessel aperture for optimal sealing of the aperture incision.

The implantable devices may be fabricated of materials which have elastic characteristics, such as superelastic materials, that allow the device to be transitioned from/to a functional state to/from a lower-profile or compressed state and back again where the device, when in the lower-profile state, has at least one dimension that is less than when in the other state. When in a lower-profile state, the device facilitates its percutaneous delivery to a target tissue site, and can subsequently be released or expanded to the functional state upon positioning at the target site. When in the functional or expanded state, the device allows for the controlled opening and closing of the incision.

The subject implantable devices may also be adapted to engage with means for securing the device to the implant site or may otherwise be configured to be self-retaining at the implant site. For example, the devices may be equipped with barbs, screws, rivets or the like to penetrate and anchor into tissue or may have a tacky surface which adheres to tissue surfaces.

The present invention further includes systems for creating the tissue apertures and delivering the implantable closure devices. The systems may include one or more instruments for cutting the incision and/or delivery and securing the closure devices at the tissue aperture. Aspects of the systems are configured to place and maintain the aforementioned implantable devices in a lower profile state. Other aspects of the systems include mechanisms to secure the device at the site. For example, the systems may include mechanisms for applying sutures, staples or clips. In other embodiments, the systems include means for presenting a positive pressure beneath the tissue surface on which the devices are to be implanted. Such positive pressure may be used to provide the necessary resistance or tension on the tissue to fixate a frame-type device at a target tissue site. For example, the positive pressure may be used to impale the tissue on to self-retention means, e.g., anchoring members, of the frame, and/or to deform the self-retention means on the back/internal side of the tissue structure. The positive pressure may additionally or alternatively be used as a backstop against undesirable penetration of the tissue, i.e., to allow a blade or other tissue cutting instrument to penetrate the tissue to form the desired incision while at the same time preventing the over-incising or puncturing to prevent the backside or opposing side of a vessel wall. Additionally, the positive pressure may be used to manipulate the engaged tissue or provide relative motion between the tissue and the implantable device/and or cutting instrumentation.

The present invention also provides methods, which include using the subject implantable devices and/or the subject systems to form access apertures within tissue structures and/or for closing those apertures. Certain of these methods include facilitating the performance of percutaneous or endovascular procedures through a controlled tissue opening.

The present invention further includes kits which include one or more implantable devices, possible of different sizes, shapes, etc., system instrumentation and other components for performing the diagnostic or therapeutic procedure, including but not limited to biological glues, fillers, sutures, clips, etc.

These and other features, objects and advantages of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. To facilitate understanding, the same reference numerals have been used (where practical) to designate similar elements that are common to the drawings. Included in the drawings are the following Figures:

FIGS. 1A-1F show various acts or steps of forming an incision within a vessel using instrumentation and according to a method of the present invention;

FIGS. 2A-2D show various acts or steps of closing an incision within a vessel by means of sutures using instrumentation and according to another method of the present invention;

FIGS. 3A-3D show various acts or steps of closing an incision within a vessel by means of staples using instrumentation and according to another method of the present invention;

FIGS. 4A-4D′ show various acts or steps of forming an incision within a vessel using a dual-puncture approach with instrumentation and according to another method of the present invention;

FIGS. 5A-5C show acts or steps of closing an incision within a vessel by means of a biologic glue using instrumentation and according to another method of the present invention;

FIGS. 6A-6M″ illustrate various examples of embodiments of implantable frames of the present invention which are used to define a tissue incision or flap to be made within a vessel or hollow tissue structure according to methods of the present;

FIGS. 7A and 7B illustrate closed and open configurations, respectively, of an exemplary frame of the present invention, as well as the relative movement between the inner and outer members of the frame;

FIGS. 8A-8I illustrate various acts or steps of a method according to the present invention for implanting the frame of FIGS. 7A and 7B to internally access a target vessel for the performance of an endovascular procedure therein;

FIG. 9 illustrates a top view of another implantable frame of the present invention having a tissue cutting mechanism integrated therewith;

FIGS. 10A-10C illustrate various acts or steps of a method of affixing the implantable device of FIGS. 6L and 6L′ to a vessel wall; and

FIGS. 11A-11E illustrate various acts or steps of a method of affixing the implantable device of FIGS. 6M-6 M″ to a vessel wall.

Variation of the invention from that shown in the Figures is contemplated.

### DETAILED DESCRIPTION OF THE INVENTION

The devices, systems and methods of the present invention are now described in greater detail in the context of vascular access applications, and more particularly, in the formation and closure of arteriotomy sites; however, such applications are not intended to limit the invention in any way, but are used solely to illustrate broadly applicable aspects of the present invention. For example, the present invention may be employed in the context of forming and closing apertures within other tissue structures such as organs and tissue walls (e.g., diaphragms). Additionally, while not specifically described or illustrated herein, the following description is not intended to exclude any commonly performed preliminary or inherent acts for preparing for the procedure, or accessing (e.g., penetrating through subcutaneous tissue) and/or closing (e.g., dressing) the site where the arteriotomy is to be formed.

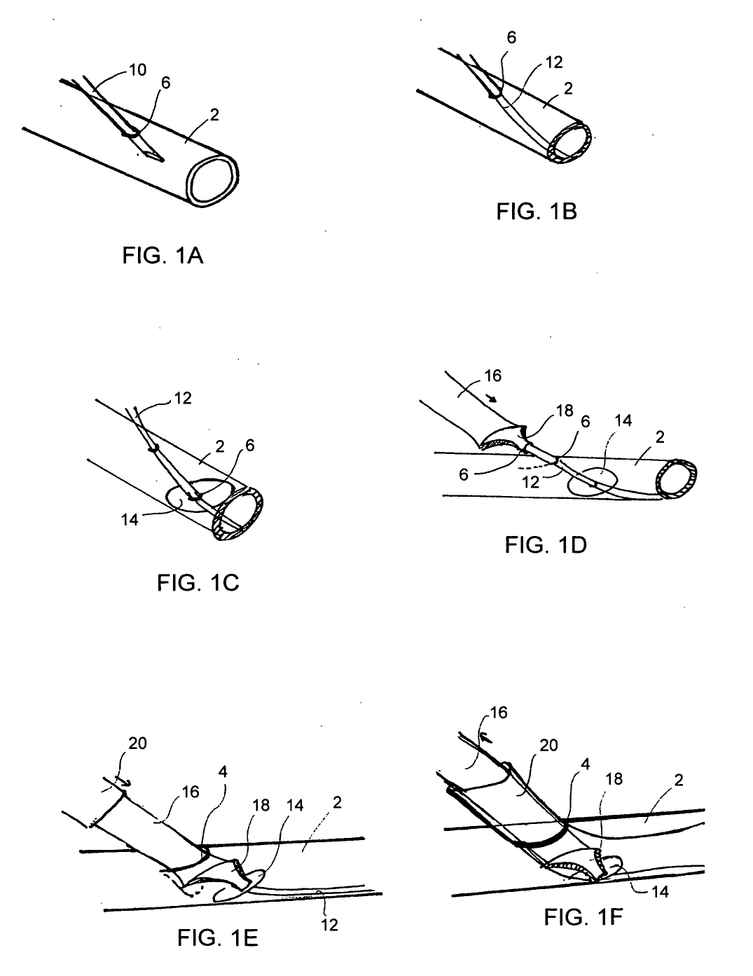

Referring now to FIGS. 1A-1F, various steps of a method according to the present invention for forming an aperture within a vessel 2 are illustrated. In order to establish access to vessel 2, a needle trocar 10 or the like is penetrated through tissue and is caused to puncture 6 the target vessel wall 2 (FIG. 1A). A guidewire 12 is then passed through needle 10 into the lumen of vessel 2, after which, needle 10 may be removed from the wound site leaving guidewire 12 behind (FIG. 1B). An expandable or inflatable back stop or stopper 14 is then delivered over guidewire 12 and expanded once within vessel 2 (FIG. 1C). Stopper 14 may be a balloon, a mesh or have any other structure which may be compressed to a reduced profile for entry into the small needle hole 6 within vessel 2, and then expanded to a greater profile so as to provide a relatively rigid or stiff back plate to protect the far side of the vessel wall from being damaged upon using an aperture forming device 16 to incise the near side of the vessel wall (FIG. 1D). The aperture forming device 16 has a working end 18 having a sharp blade or cutting edge for incising the vessel wall 2. The blade or edge may be provided with a shape or curvature of that desired for the incision 4 to be formed. For example, a crescent, U, or C-shaped blade may be used to form a flap or the like that fans distally of the central or guidewire entry site 6. The length and/or radius of curvature of the blade, and thus of the incision, may be such that, when the flap is separated (pushed or pulled) away from the vessel, it forms an aperture sufficiently sized and shaped for the delivery of instrumentation for performing the endovascular diagnostic or therapeutic procedure within the vessel. While not required, a sheath 20 may be employed through which the procedural instrumentation (e.g., cannulas, catheters cutting tools, stent placement catheters, angioplasty catheters, scopes, etc.) may be delivered (FIG. 1E) subsequent to removing cutting instrument 16 and stopper (FIG. 1F) from vessel 2. As such, the size, shape and location of the aperture through which the procedural instrumentation 20 enters vessel 2 are controlled.

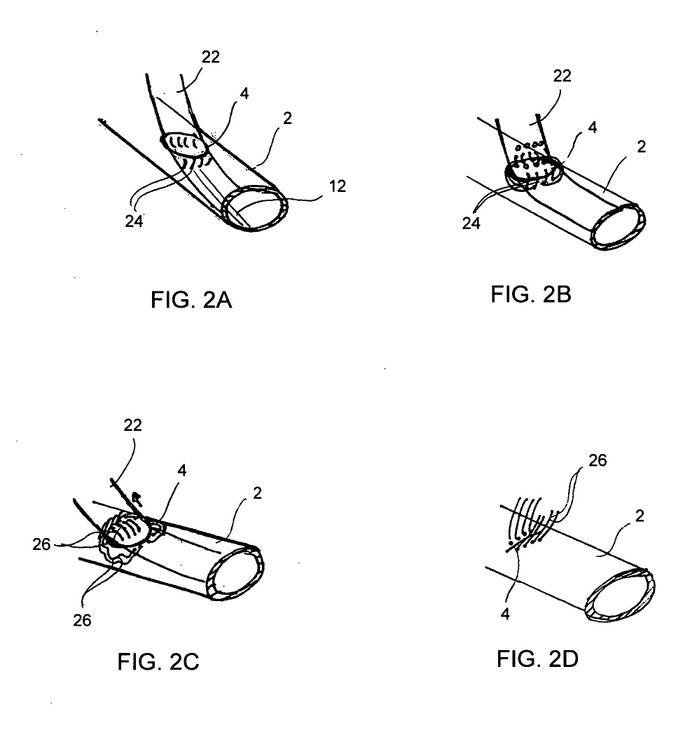

After the endovascular procedure is completed, aperture 4 may be closed using various modalities including suturing, stapling, clipping, gluing, etc. One such suturing modality of the present invention is illustrated in FIGS. 2A-D. A suturing instrument 22 with preloaded suture needles 24 is provided and delivered over guidewire 12 to the entry site or aperture 4 (FIG. 2A). Once properly positioned, needles 24 are deployed from instrument 22 to within the vessel wall 2 at opposite sides of incision 4 (FIG. 2B). The sutures 26 are then tightened to snugly oppose the edges of aperture 4, and instrument 22 is removed along with needles 24 leaving behind sutures 26 (FIGS. 2C and 2D).

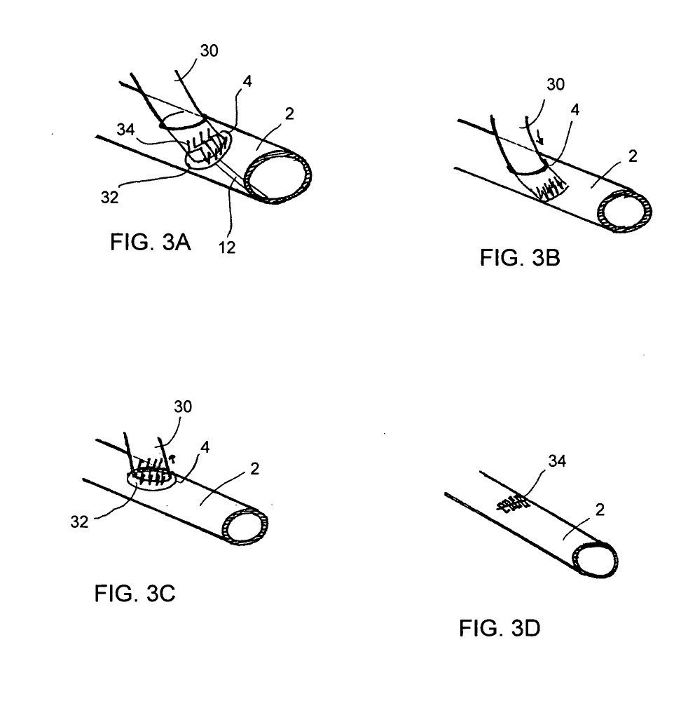

Alternatively, a stapling modality of the present invention, as illustrated in FIGS. 3A-3D, may be used to close aperture 4. A stapling instrument 30 with preloaded staples 32, typically provided in a cartridge, is provided and delivered over guidewire 12 to the vessel entry site or aperture 4 (FIGS. 3A and 3B). An anvil 32 or the like (such as stopper 14 of FIG. 1) may be used as a back stop to deform the staples 34 once deployed from instrument 30 (FIG. 3C). The implanted staples 34 straddle across the opposing edges of aperture 4. (FIG. 3D).

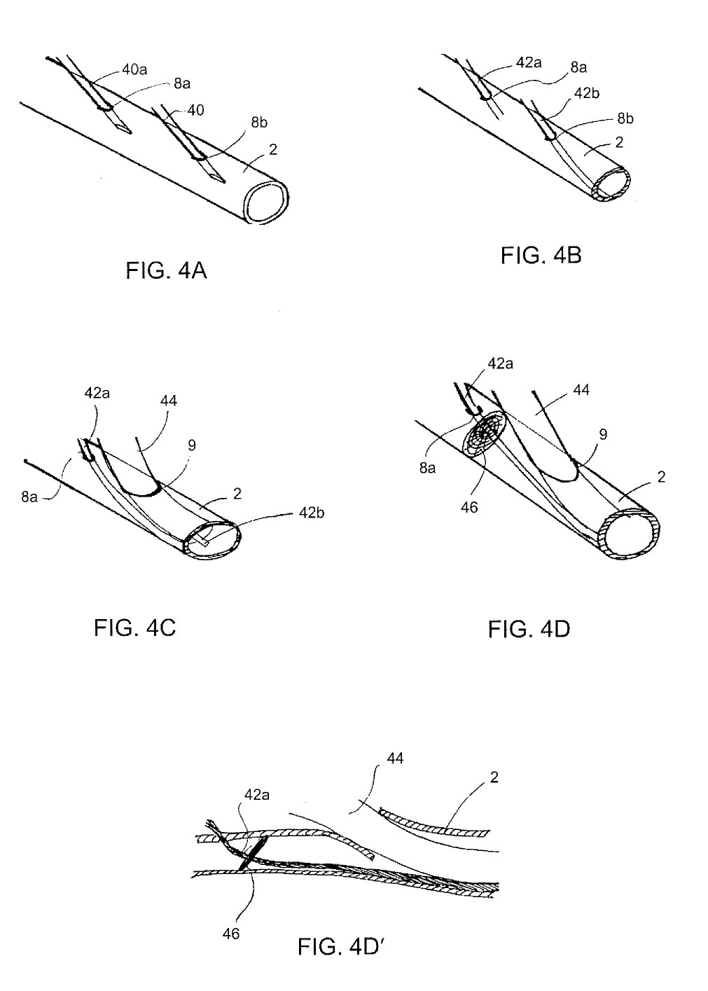

Another method of the present invention is illustrated in FIGS. 4A-4G in which a two-point approach to performing an endovascular procedure according to the present invention. Here, two needle trocars 40a, 40b are used to penetrate vessel 2 at a spaced-apart distance from each other (FIG. 4a) to establish two entry sites 8a, 8b from about 0.5 to about 1.5 inches apart. Then, a separate guidewire 42a, 42b is delivered through each needle trocar to within vessel 2 (FIG. 4B). By means described above with respect to FIGS. 1C-1F and using upstream (relative to blood flow) guidewire, here guidewire 42b, an aperture 9 may be formed. A sheath 44 is then positioned within aperture 9 for delivery of instrumentation to be used in the diagnostic or therapeutic endovascular procedure to be performed (FIG. 4C). Optionally, guidewire 42a is used to deliver a debris collector 46 to be positioned downstream of the site of therapy and of sheath 44 in order to filter out any embolic, thrombogenic or other foreign material that may be present within the downstream blood flow (FIGS. 4D and 4D′). Debris collector 46 may be a mesh or other material commonly used for embolic filters.

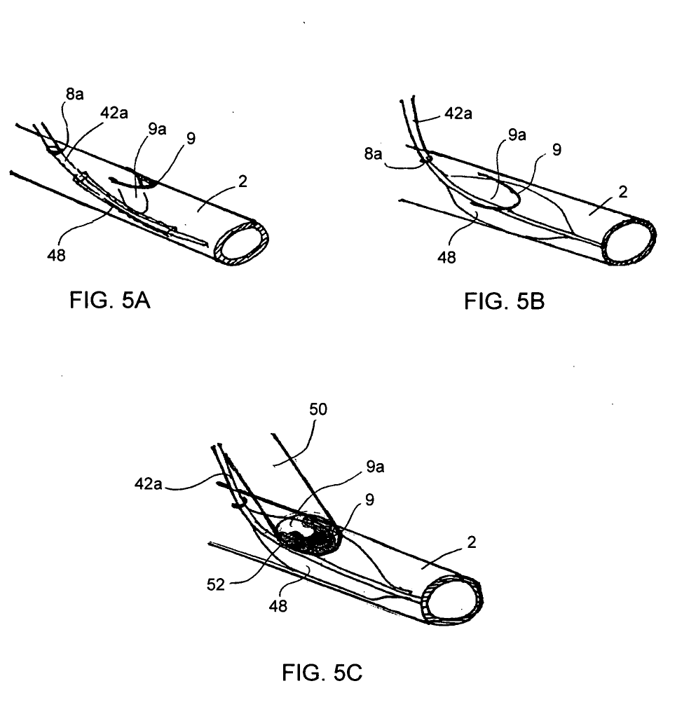

FIGS. 5A-5C in turn illustrated acts or steps of a procedure according to the present invention in which the second or downstream vessel entry site 8a is used in closing and sealing aperture 9. In this method, an expandable or inflatable member 48, such as a balloon, is delivered over guidewire 42a to within vessel 2 (FIG. 5A). Balloon 48 is positioned under or aligned with aperture 9 and then inflated to cause the aperture flap 9a to be repositioned such that its edges align with the vessel wall 2 (FIG. 5B). A glue or sealant delivery tube 50 is then inserted within the wound over aperture 9 and a sealant material 52 is delivered over the incision 9 (FIG. 5C). The material may then be actively cured with heat or light (not shown), or may otherwise be self-curing.

In addition to the above described tools and techniques for creating and closing incisions and apertures within a tissue wall, the present invention also includes novel implantable devices which provide even greater control in the formation and closure of such apertures. These devices are structures, templates or frames having at least one border or edge for defining shape and/length of the incision to be made within the target vessel or hollow tissue structure. In many variations, the devices have substantially closed perimeters or define an aperture having the desired shape and size of the incision. Mechanical and/or material characteristics of the frames facilitate and control the relative motion that can be imposed on the apposing tissue edges or “flaps” formed upon making an incision. For example, mechanical features, e.g., cusps, within the frames may be employed to decouple the relative motion between opposed tissue flaps and/or to provide strain relief to the frame sides so that they are easily separable for passage of instrumentation therethrough. Additionally, the frame material may have characteristics which bias the frame to closed or planar condition to facilitate proper apposition of the tissue edges thereby enhancing healing of the incision.

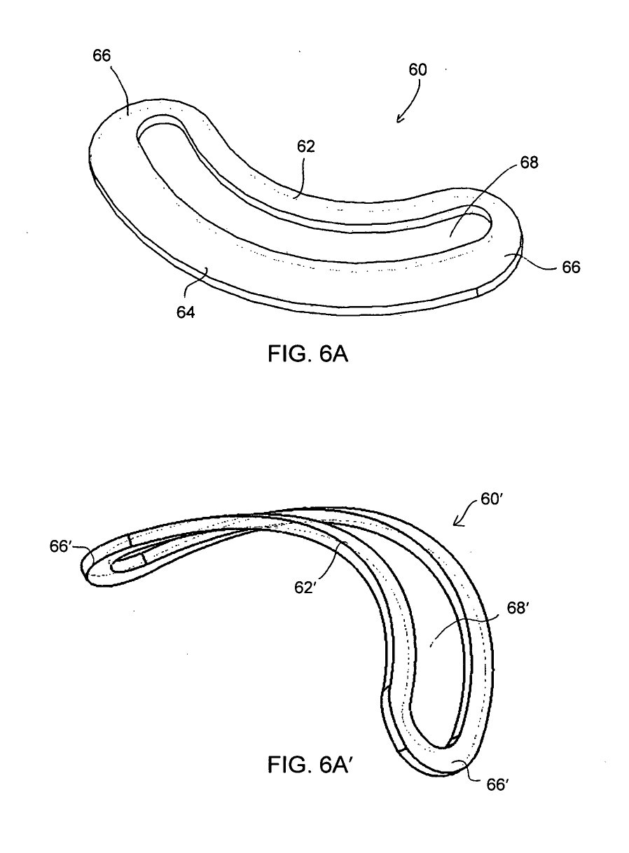

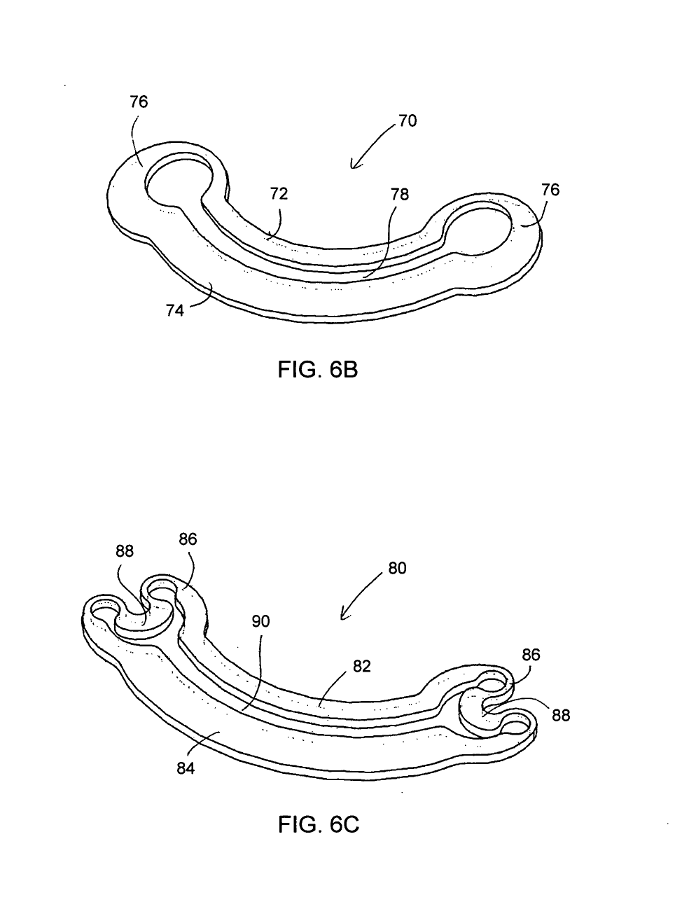

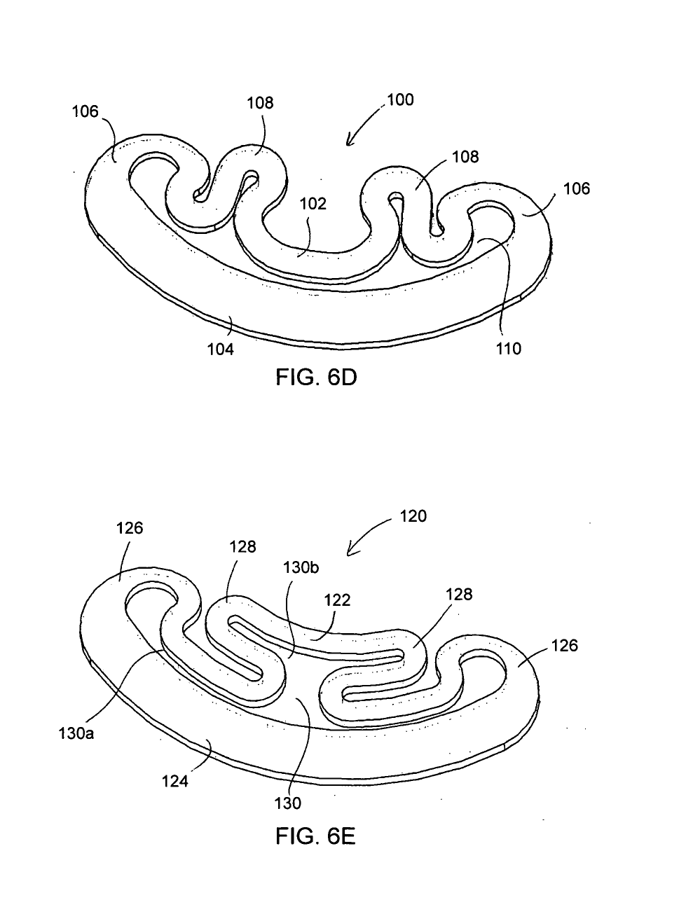

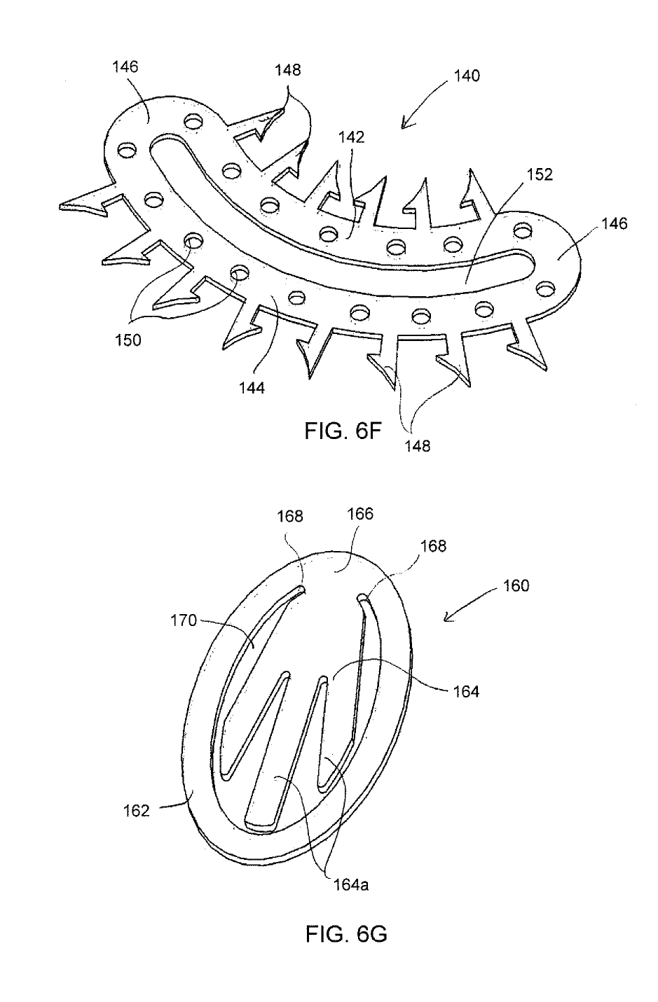

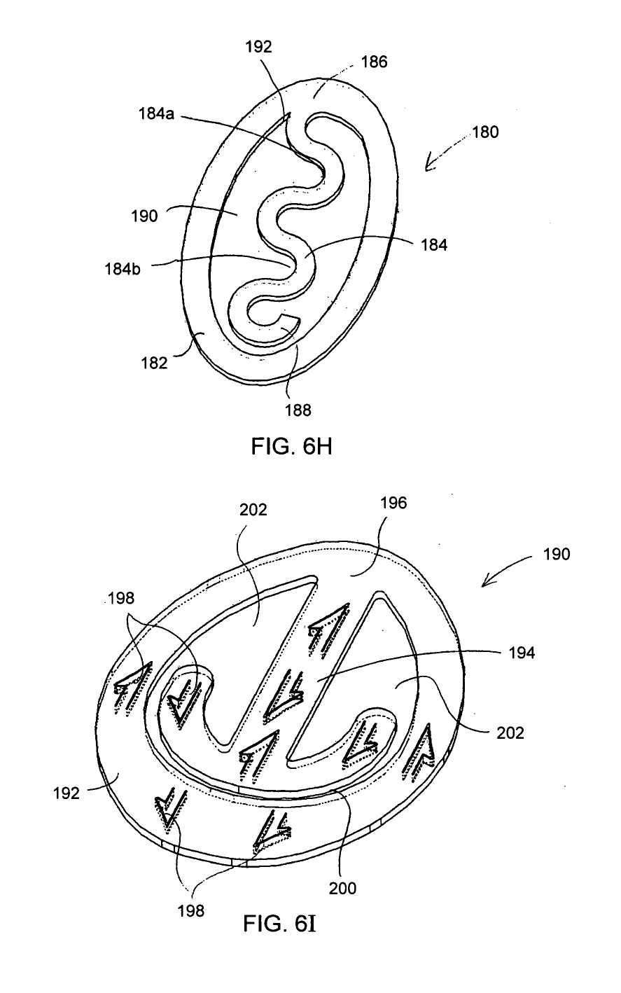

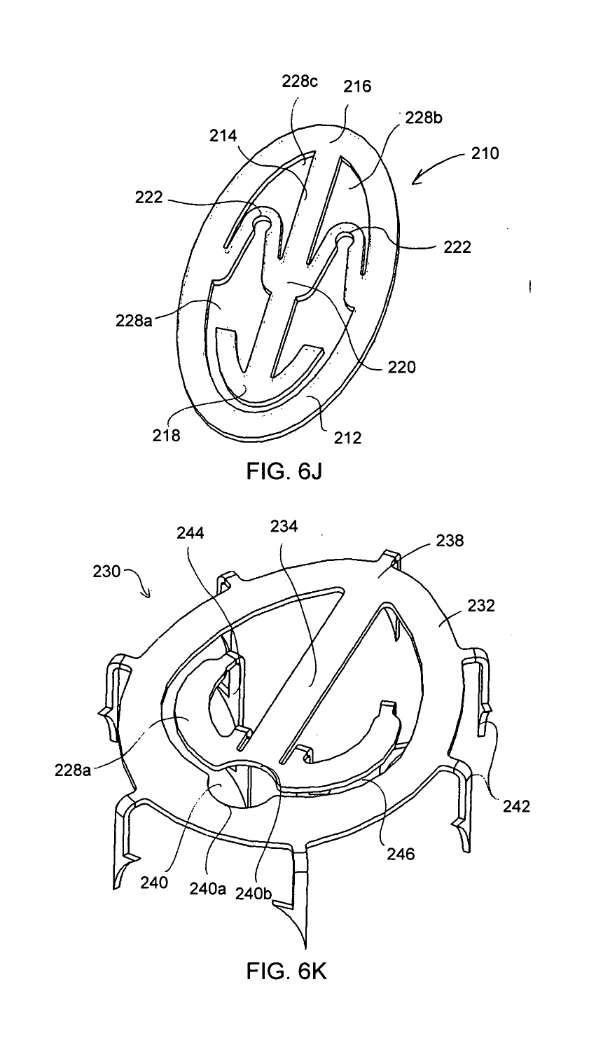

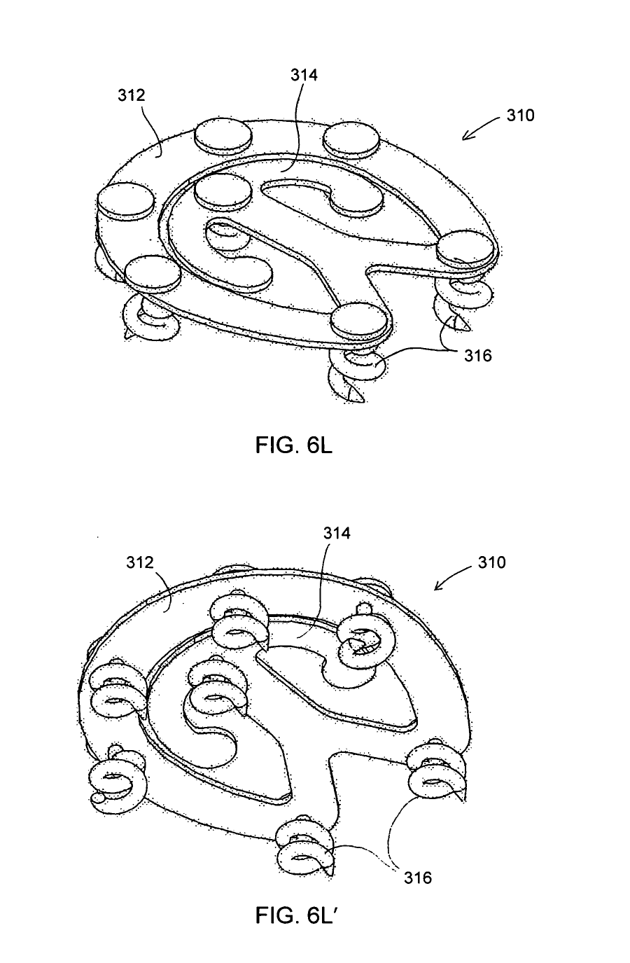

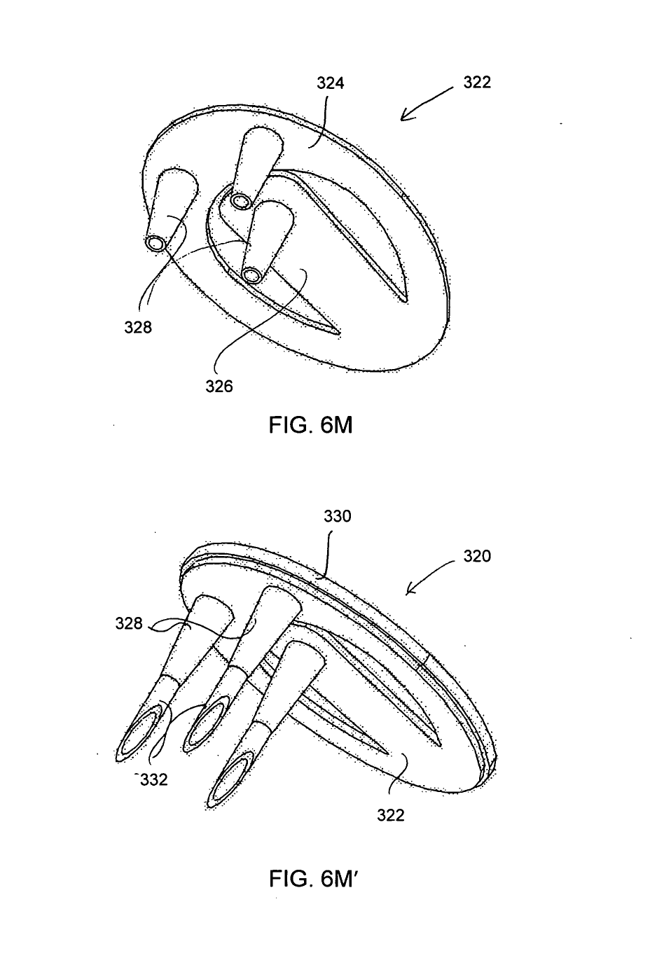

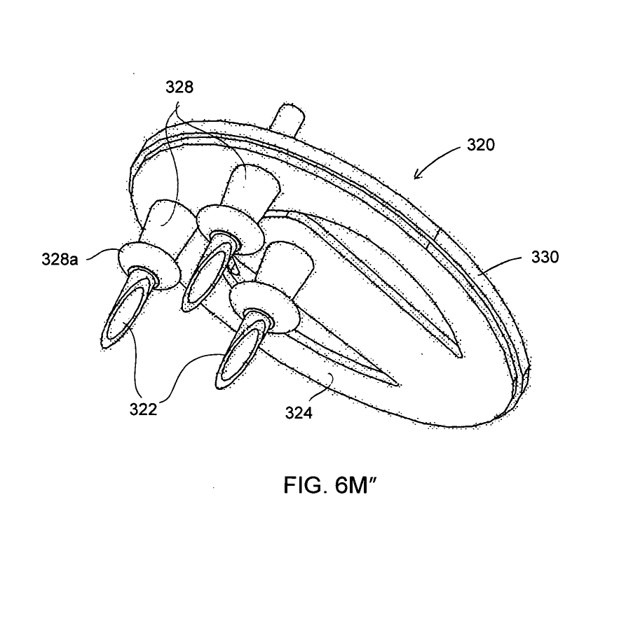

Various embodiments of the subject frames are illustrated in FIGS. 6A-6J. FIGS. 6A-6F show frames having an outer profile having a crescent or arc shape while FIGS. 6G-6J show frames having an outer profile having an elliptical or circular shape. However, the frames may have any outer profile shape (e.g., circular, square, rectangular, kidney, etc.), preferably one that is suitable for the bodily location into which a frame is implanted. The frames of the present invention have inner profiles which define one or more gaps, spaces or apertures through which an incision (or incisions) may be made into the tissue structure onto which the frame is positioned. The incision forms one or more tissue flaps, slits, apertures, or the like through which a procedure may be performed. More specifically, the incision allows catheters and the like to be delivered within a vessel for performing an endovascular procedure.

The incisions may be formed by conventional scalpels or other tissue incising instruments or may otherwise be made with tissue cutting instruments of the present invention. With the latter approach, a tissue cutting instrument may be employed prior to or after the aforementioned frames are implanted, where the instrument has a distal blade or the like having a configuration or shape which provides an incision having the desired profile of the tissue flap to be formed. The cutting instrument may be incorporated into a system or integrated with other components to form a system for performing the methods of the present invention.

Depending on the application at hand and the size (diameter) of the vessel or tissue structure which is being incised, the framed aperture(s), if rounded, annular or scalloped, has a radius of curvature in the range from about 2 mm to about 5 mm and an arc length in the range from about 6 mm to about 15 mm; however these values may be lower or greater than the stated ranges. For example, larger apertures may be preferred for cannulating an aorta or vena cava whereas a smaller opening may be desired for percutaneous access through a femoral artery or vein.

The frames are generally planar and may be completely flat or have a curvature about their planar aspect to match that of the outer surface of the vessel against which they are placed. To this end, the frames may be made of a rigid or semi-rigid material having a preformed curvature substantially matching that of the vessel over which they are to be positioned. In other embodiments, the frames may be made of a flexible or semi-flexible material so as to be conformable to the curvature of the vessel over which they are placed. The preformed or user-formed curvatures of the frames structures have radii of curvature in the range from about 4 mm to about 12 mm matching that of the outer wall of the vessel to be incised.

The frames may be made from any biocompatible material to provide the desired flexibility or rigidity. Exemplary frame materials include but are not limited to metals, such as stainless steel and Nickel-Titanium, and polymers, including bioresorbable or biodegradable polymers such as poly(lactic acid) (PLA), poly(glycolic acid) (PGA), poly(lactic-co-glycolic acid) (PLGA) and the like.

The frames are configured to have portions or segments which are movable relative to each other whereby one or more of the frame portions is able to be positioned or moved “out of plane” from the remainder of the frame structure where relative movement between frame portions may be somewhat “jaw-like.” For example, the vessel wall or tissue flap(s) formed by an incision made in a vessel may, by movement of a frame portion, be able to be moved outwards or upwards out of plane from the vessel. Alternatively, the flap(s) may be movable to within the lumen of the vessel where it is also out of plane. As used herein, the phrase “out of plane” is not limited to flat surfaces or planes but also includes surfaces or planes defined by structures which have a non-flat surface, such as a curved or rounded surface. As such, a curved frame of the present invention also defines a (curved) plane just as a flat frame does.

To accomplish the relative motion of the frame portions, the frame structure has one or more flexure points. Such flexure points may be defined by a joint or hinge mechanism. Alternatively, the material characteristics and shape of the frame structure or portions thereof, such as preformed cusps within the frame structure itself, may define one or more flexure points or “living hinges” to allow separation of opposing or adjacent frame portions.

Depending in part on the shape of the interacting frame portions and in part on the shape and number of the incisions made with the frame acting as a template for such incisions, the manner in which the frame portions are separable and the resulting configuration of the vessel opening will vary. For example, frames having crescent or kidney shaped structures (see FIGS. 6A-6F) provide a jaw-like action when operably moved, i.e., opened, spaced or separated from each other. On the other hand, the subject frames having a more annular configuration which creates a tissue flap that functions more like a lid or a portal. Using any of the described frames, the incisions are typically annular or define a conic arc, extending from end-to-end less than 360°, and most typically extend no more than about 180°. In other embodiments, the frames may be configured to provide straight incisions having a desired length.

Additionally, the material characteristics of the frame or its hinged or cusped portions may be biased open or closed. In the former variation, the frame would be forced closed to define the aperture through which a tissue incision is to be made, and then allowed to open whereby separation of the sides of an incision form an open gap or flap that does not require other means, e.g., sutures, to remain open during the procedure to be performed. When closing the incision, the frame is bent so that the frame portions remain in plane with each other in order to establish permanent apposition between the incision sides to facilitate healing. Alternatively, the separable frame portions may be interconnected together, e.g., by suturing, tying clipping, etc., to maintain apposition between the incision edges. With those embodiments having frames which are biased closed, active means may have to be used to keep the incision open during the procedure. However, such may not be required as the instrumentation used to perform the procedure may hold the incision open. Conversely, no bending or other means may be necessary to hold the biased-closed frame portions together to obtain the desired apposition between the incision edges upon closure of the wound. Shape memory metals such as NITINOL (NiTi) are particularly suitable for providing the various frame characteristics just described. With reference to FIGS. 6A-6J, these and other features, characteristics and abilities of the subject frames are further described.

Frame 60 of FIG. 6A has a flat, planar structure having inner or concave frame side 62, outer or convex frame side 64 and ends, joints or cusps 66 which collectively define an aperture 68 having a simple arc shape. Aperture 68 provides a template for the incision to be made in the vessel over which frame 60 is positioned. FIG. 6A′ illustrates a similarly arc-shaped frame 60′ with opposing frame sides 62′, 64′ and frame cups 66′ collectively defining an arc-shaped aperture 68′; however frame 60′ has a curved or bowed structure, rather than a flat structure, having a radius of curvature substantially matching that of the vessel wall.

Frame 70 of FIG. 6B has similar inner and outer frame sides 72, 74, however, cusps 76 define an inner aspect where the collective frame structure defines an arced opening 78 having dumbbell-shaped ends. Frame 80 of FIG. 6C has inner frame side 82 and outer frame side 84 similar to those just described; however, cusps 86 are double-pronged to define inwardly extending tabs 88. Collectively, this frame defines an arced aperture having bone-shaped ends. Such a configuration facilitates decoupling the mobility of the resulting convex-shaped tissue flap and the apposing concave tissue flap.

Frame 100 of FIG. 6D has a regular convex outer side 104 and cusps 106, however, inner frame side 102 has an undulating pattern to further define a second pair of cusps 108. As such, frame aperture 110 provides the option of forming an incision with a wider or narrower radius of curvature. More specifically, if an incision is made with ends terminating in outer cusps 106, a wider radius of curvature is provided. On the other hand, if the incision ends are traced within inner cusps 108, a vessel incision having a narrower radius of curvature is formed.

Frame 120 of FIG. 6E also has a convex-shaped outer frame side 124 and hairpin-shaped outer cusps 126 with an inner frame side 122 having a brief serpentine pattern. As such, frame 120 defines an aperture 130 which also provides for the choice between two incisions; however, here the incisions would have the same radius of curvature and vary in arc length only. More specifically, an incision traced within portion or sub-aperture 130a defined by outer cusps 126 is longer than that which is traced within portion or sub-aperture 130b defined by inner cups 128.

Frame 140 of FIG. 6F has the same simple frame shape of that illustrated in FIG. 6A; however, the outer edges of concave and convex frame sides 142, 144 are provided with a plurality of radially extending barbs or prongs 148 for piercing and anchoring into tissue. These barbs can be bent out of plane as to better engage tissue. Frame 140 may be further equipped with a plurality of through-holes 150 spaced apart along the frame sides to provide anchoring points for sutures or tacks to additionally or alternatively anchor the frame to the vessel wall or to attach the frame to the frame delivery device. Further, holes 150 may be used to bridge or straddle a suture, wire or the like across frame aperture 152 in order to permanently close the vessel incision, to maintain the opposing frame sides 142, 144 within plane and to maintain the incision edges in apposition with each other. For those frame embodiments without prongs and/or anchoring holes, the frame structure may be held in place at the operative tissue site with sutures, clips, glue or the like.

FIGS. 6G-6J illustrate other variations of the subject frames which have an outer frame structure and an inner frame structure which is hinged to the outer frame structure. Typically only one hinge is provided however more than one hinged point may be employed. The outer frame structure is substantially annular, having either an elliptical, egg or circular shape. The ring or band of material forming the outer frame structure may have a substantially constant shape and width to provide similarly shaped inner and outer edges. The inner frame structure may have any suitable configuration (i.e., shape, pattern or surface area) which provides spacing or a gap between it and the outer frame structure to define an aperture therebetween. Accordingly, one or more incisions may be made within the aperture to form one or more tissue flaps having a desired configuration, i.e., shape, length, pattern etc. within the parameters of the frame aperture. For example, an incision may be made which is defined by either the inner edge of the outer frame structure, the outer edge of the inner frame structure, or alternatively defined by selectively using aspects of one or both edge surfaces, or may otherwise be free form by not using any of the frame edges as a template for the tissue flap to be formed.

Referring to FIG. 6G, a frame 160 having an elliptically-shaped outer frame 162 defining and a forked shape inner frame 164 is provided. Inner frame 164 is attached or hinged to outer frame 162 at the end opposite of tines or fingers 164a. The interface between the inner and outer frames at hinged portion 166 defines cusps 168. A tissue incision extending from one cusp to the other in the space between the inner and outer frames would provide an elliptically-shaped (or similar) tissue flap; however

Frame 180 of FIG. 6H has a similarly shaped outer frame 182 structure with an inner frame 184 having an undulating or serpentine pattern. One end of inner frame is attached to outer frame 184 at hinge point 186. The aperture 190 defined between the inner and outer frame structure provides many options as to the length, shape and number of tissue incisions that could be made. For example, a single incision may be made by tracing a scalpel or other cutting instrument along the edge of inner frame 184 from cusp 192 to free end 188 to form a scalloped shape incision. Alternatively, an incision extending along the edge of a single undulation 184a of inner frame 184 may be made. A second similar incision may be made in another undulation 184b to provide two tissue openings or flaps, etc. It is noted that the free or unattached end 188 of the inner frame may have a loop or hook shape to which a suture, clip, wire or the like may be engaged in order to maneuver (push or pull) the inner frame relative to the outer frame, or to secure the inner and outer frame structures to each other when permanently closing the tissue opening.

Frame 190 of FIG. 6I has an outer frame structure 192 having an egg-shaped outer profile and an inner frame structure 194 having a modified T-shape and attached to outer frame structure 192 at hinge point 196. The inner and outer frame structures define an aperture therebetween in part in the form a gap 200. The aperture further includes ancillary portions 202 which allow for an incision having an extended arc length. As with the other frame embodiments, any suitable incision or incisions may be made within the aperture space. The frame structures 192, 194 are optionally provided with barbs or spikes 198 for anchoring to tissue against which they are engaged. The barbs may be pre-cut at various locations with in the frame structure, as illustrated, and then manually flared at the time of implant. This allows the option of using any number of the pre-cut barbs or none at all. Alternatively, the frame may be provided with pre-flared barbs (see FIG. 6K).

FIG. 6J illustrates a frame 210 of the present invention having an elliptical outer frame structure 212 and an elongated inner frame structure 214 having one end attached to outer structure 212 at hinge point 216 and an opposing T-shaped free end 218. Extending from a central portion 220 of inner structure 214 across the minor axis of outer structure 212 are two opposing frame segments 222. Both frame segments 22 are affixed to inner and outer frame structures. As such, three separate frame apertures 228a, 228b and 228c are defined. However, any other number of bridge segments (i.e., one or more than two) may be provided to define any number of frame apertures (i.e., two or more than three). The apertures may be similar or different from each other, whereby their relative shapes are determined in part by the shape of the bridge segments 222 and the configuration of free end 218 of the inner frame (amongst other portions of the frame). Here, frame segments 222 define cusps which extend toward the hinged end 216 of inner frame 214. As such, a tissue incision made within aperture 228a extending from cusp to cusp and around free end 218 would provide a flap which has a potential maximum surface area smaller than the surface area defined by the inner edge of outer frame structure 212.

FIG. 6K illustrates another frame 230 having an elliptical outer frame structure 232 and a T-shaped inner frame structure 234 hinged thereto, which are similar in configuration to the corresponding structures of frame 190 of FIG. 6I. Frame 230 has a couple of additional features including an aperture 240 defined by indents or cutouts 240a of outer frame 232 and 240b of inner frame 234 at a location opposite flexure point 238. Hole 240 allows the frame to be easily translated over a guidewire to a target tissue site and also facilitates use of a finger or instrument to grasp inner frame 234 in order to pull up or push down on the flap created by an incision to be formed in the gap 246 between the frame structures. As with the frame of FIG. 6I, optional prongs 242 and 244 may be provided on the outer and inner frame structures, respectively, for further securing the frame within the vessel wall. Here, barbs 242, 244 extend radially outward and inward of the frame members and are shown in a flared or active position (i.e., outside the plane defined by the frame).

FIGS. 6L and 6L′ illustrate a frame 310 an elliptical outer frame structure 312 and an inner frame 314 hinged thereto. Here, the anchoring means are coil screws 316 which are deliverable through screw holes (not visible) within one or both of the inner and outer frame structures. Upon placement of frame 310 at a target tissue surface, one or more coil screws 316 are rotated through the tissue exposed through the holes. The diameter and pitch of the screws is wide enough to prevent the coil screws from backing out of the vessel when subject to intravascular forces. In certain embodiments, the screw diameter is within the range from about 1 to about 3 mm and a pitch less than about 45°. They have a length that is short enough such that they only marginally penetrate the opposing back wall of a vessel into which frame 310 is implanted, as explained in greater detail below.

FIGS. 6M-6M″ illustrate another variation of the subject frames in which a two-piece assembly 320 includes a bottom frame plate 322 and a top frame or back plate 330. Each of the plates 322, 330 has an elliptically shaped outer frame 324 and a hinged inner frame 326 (only those of bottom plate 324 are visible). Extending from the bottom surface of the inner and outer frames of bottom plate 322 is a plurality of open sleeves or receptacles 328 configured to matingly receive corresponding pins 332, which are shown positioned through holes (not visible) provided within top plate 330. Sleeves 328 are sized and/or made of a material that enables a press-fit engagement with pins 322 such that the pins are not easily removed from the frame. Sleeves 328 further configured such that their distal portions 328a are caused to buckle upon proximal pulling of top plate 330, thereby sandwiching the tissue wall against the underside of bottom plate 322 and securing the frame assembly 320 within the implant site.

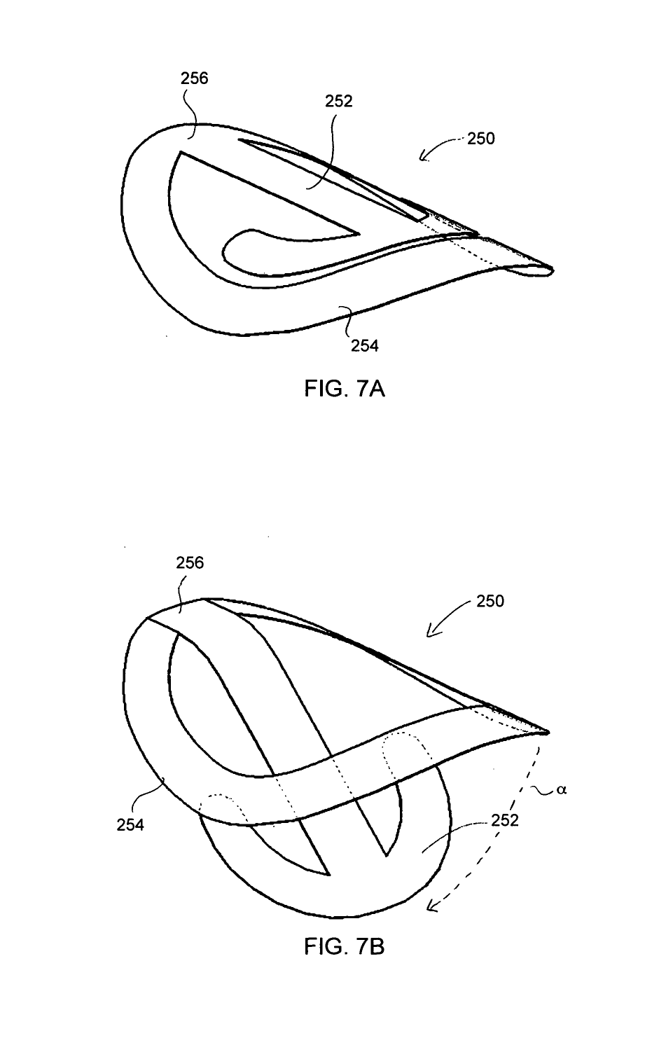

FIGS. 7A and 7B illustrate an exemplary frame embodiment 250 of the present invention having an inner frame 252 and outer frame 254. FIG. 7A shows frame 250 in the configuration which it would have when operatively engaged with the outer surface of a vessel wall upon securement thereto. FIG. 7B illustrates the relative movement of the inner and outer frame structures in operative use. In particular, inner frame 252 has been pushed downward out of plane relative to outer frame 254; however, the inner frame may pulled upward relative to the outer frame and/or the outer frame may be moved in either direction relative to the inner frame.

The extent to which the inner frame member (and/or outer frame member), and thus the tissue flap, can be opened or spaced from the outer frame or surface of the vessel wall to which a subject frame is positioned depends at least in part on the physical properties of the material used to make the frame and the thickness of the frame. Typically, the maximum angle α to which the frame members can be separated from each other without inducing permanent deformations to the frame is highly dependent upon the frame material and thickness. For example, for NITINOL based frames having a frame thickness of about 0.01 inches, the angle α is the range from between about 45° and about 90°.

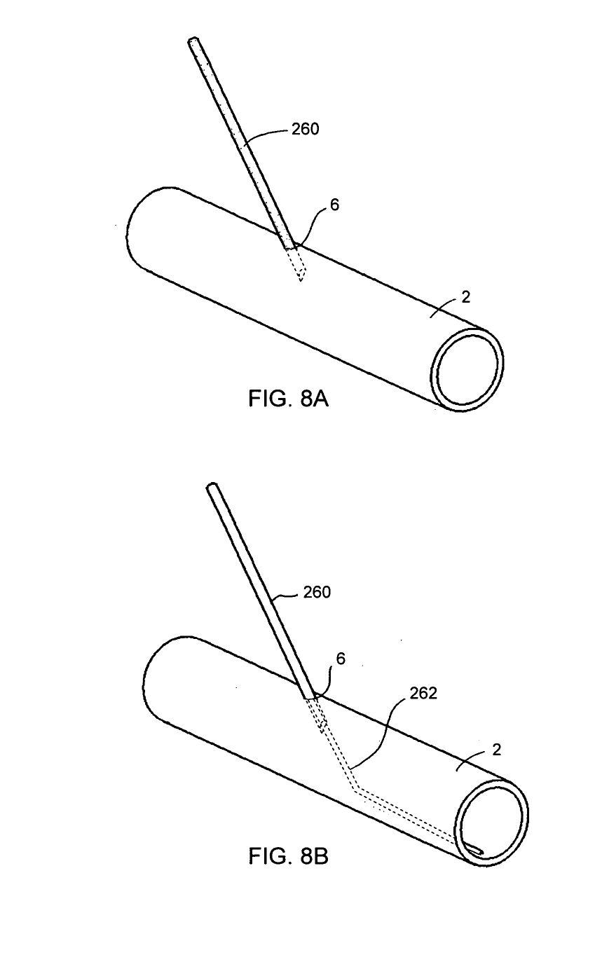

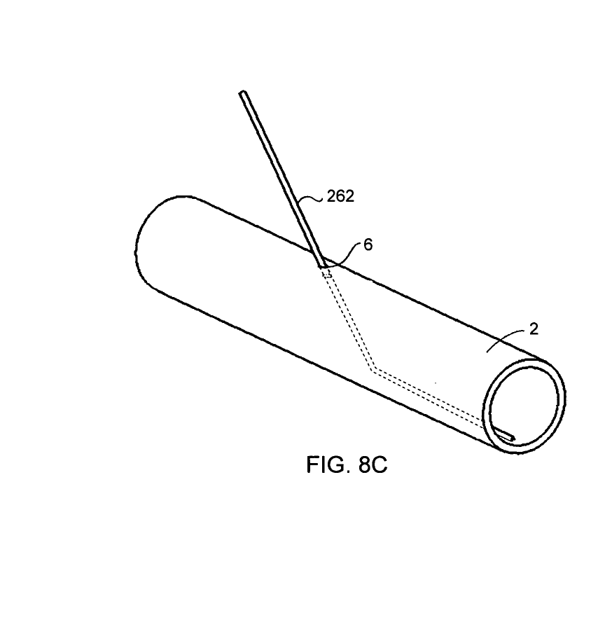

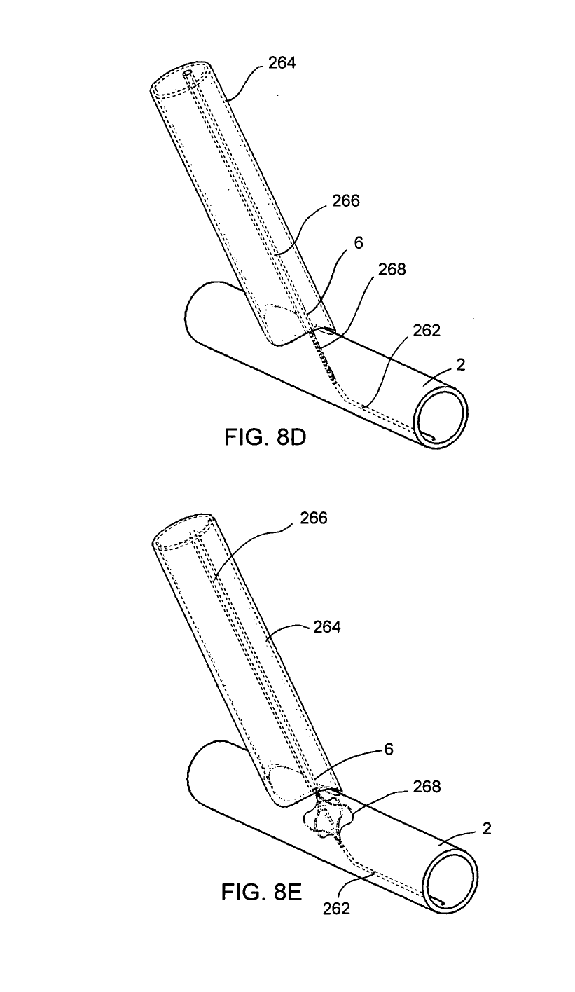

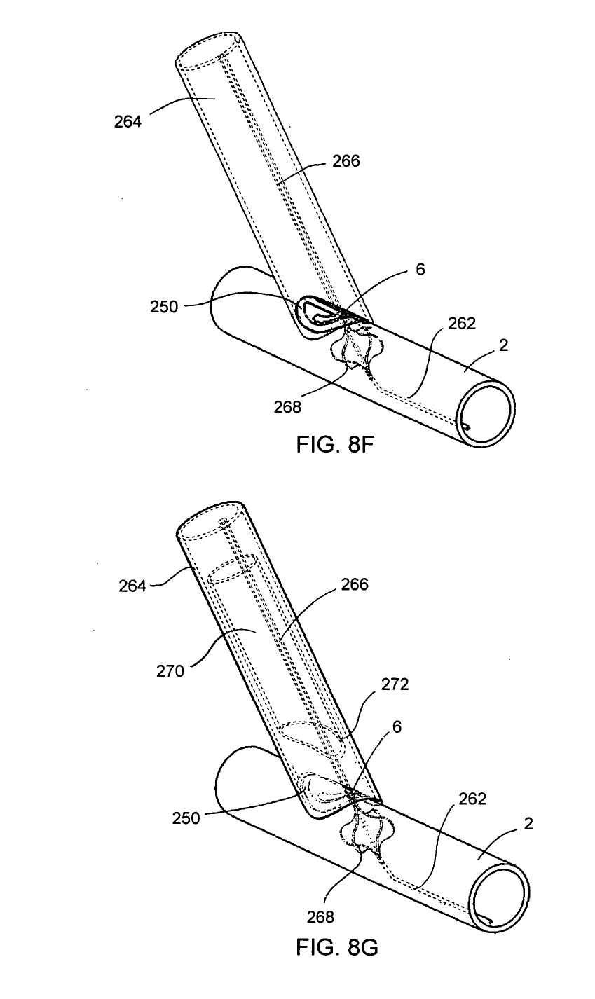

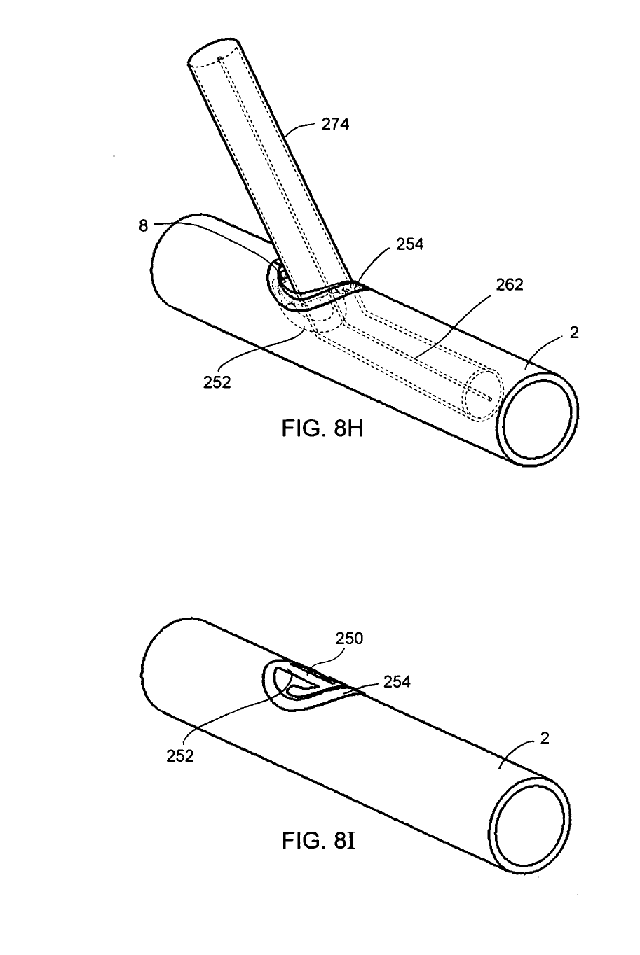

With reference to FIGS. 8A-8I, a description is now provided of a method which includes implanting and using a subject frame for the controlled formation and subsequent closure of an arteriotomy within a vessel 2 for the purpose of allowing controlled access to the vasculature for performing an endovascular procedure.

As shown in FIG. 8A, hypodermic needle 260 is used to establish an initial puncture or entry site 6 within vessel 2. A guidewire 262 is the advanced through needle 260 until a distal portion of the guidewire resides within vessel 2 (FIG. 8B), after which, needle 260 may be removed from the wound site leaving behind guidewire 262 (FIG. 8C). Next, an arteriotomy instrument of the present invention is delivered over guidewire 262 toward the vessel entry site 6 (FIG. 8D). The arteriotomy instrument may include various integrated components including a delivery tube or shaft 264 having a lumen therein through which other components or instruments are delivered. For example, an optional stopper instrument 266 is delivered through shaft 264 and over guidewire 262 through entry site 6 until a distally positioned stopper mechanism 268 (delivered in a low-profile or undeployed or unexpanded state) is positioned within the vessel lumen 2 (FIG. 8D). Stopper mechanism 268 is then deployed or expanded against the inside wall of the vessel lumen 2 at puncture site 6 (FIG. 8E). The stopper mechanism may have any suitable configuration which can be delivered in a low profile state and then transitioned into a larger profile to function as a shield to protect the interior of the vessel and/or as a back stop or anvil. As such, stopper mechanism 268 may be made of a mesh, metal braid, polyer or superelastic material (e.g., NITINOL), balloon, etc.

As shown in FIG. 8F, the arteriotomy instrument may also be adapted to deliver and implant a subject frame 250 on vessel 2. Frame 250 may be delivered through shaft 264 operatively carried by another instrument, or may be operatively carried at the distal end or edge of shaft 264. When using rigid frames, the former variation requires the diameter of shaft 264 to be at least as large as that of the frame. However, with semi-flexible or flexible frames that are able to be folded or compressed to a lower profile, shaft 264 may have a smaller diameter. With variations of the arteriotomy instrument configured to carry frames at their distal ends, the diameter and shape (i.e., footprint) of shaft 264 may be substantially similar to that of annular-type (e.g., circular or elliptical-shaped) frames. With crescent or arc-shaped frames, the cylindrical wall of shaft 242 has a similar sized radius of curvature so that the frame is able to be carried on the distal end (e.g., on the distally facing edge) of shaft 264 without affecting the profile thereof.

Referring again to the drawings, with frames having a guidewire thru-hole (as described with respect to the frame embodiment of FIG. 6K), the frames are easily translated over guidewire 262 and can be precisely positioned relative to puncture site 6. Stopper mechanism 268 provides a positive pressure or opposing force by which to accurately locate the vessel and advance and position frame 250 on the vessel. Furthermore, with frames having barbs or prongs for penetrating tissue, stopper mechanism 268 may be used as anvil with which to deform, bend, or engage the barbs once penetrated to within the vessel lumen. Stopper 268 may be further employed as a back stop for a blade or other incision-forming instrument, thereby preventing injury to the back wall of the vessel.

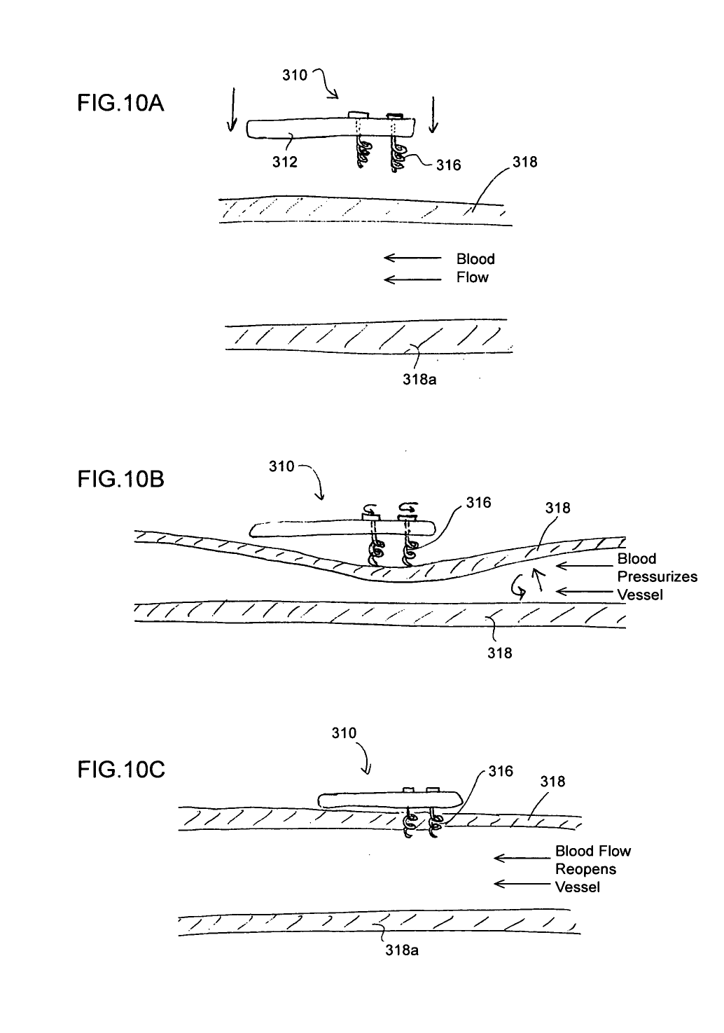

With certain embodiments of the implantable frames, a stopper mechanism or backstop may not be necessary in order to safely place and secure the frames at the implant site. For example, the frame embodiment 310 of FIGS. 6L and 6L′ does not require use of a stopper within the vessel interior. As illustrated, in FIGS. 10A-10C, frame 310 is delivered and positioned on and parallel to a surface of a blood vessel 318. Without a stopper mechanism, as the coil screws 316 are rotated into vessel wall 318, vessel wall 318 is initially compressed thereby closing off normal blood flow (see FIG. 10B). Continued rotation and downward pressure on coil screws 316 causes them to penetrate vessel wall 318, possibly as well as the opposing vessel wall 318a, depending on their effective length. In any case, the lengths of the coil screws 316 are such that that any penetration into the opposite wall would be nominal with the penetrated length being easily retracted from the opposite tissue wall by the blood pressure created upon removal of downward pressure on vessel 318. Their lengths, however, as well as their diameters are such that they are not removable from the top tissue wall 318 by normal blood flow alone.

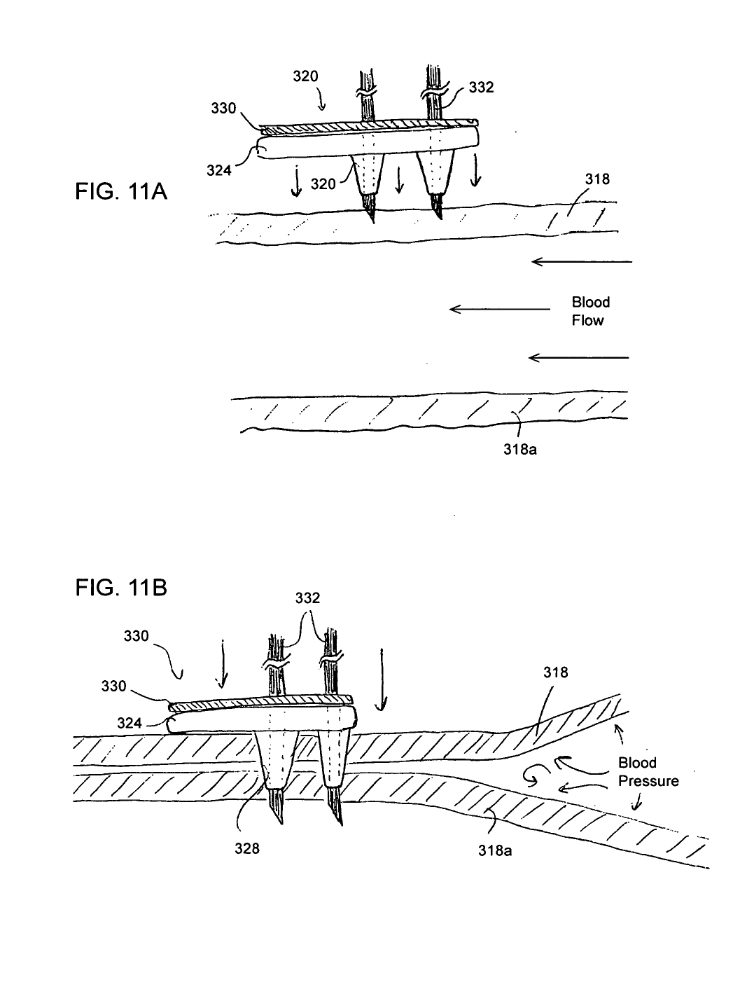

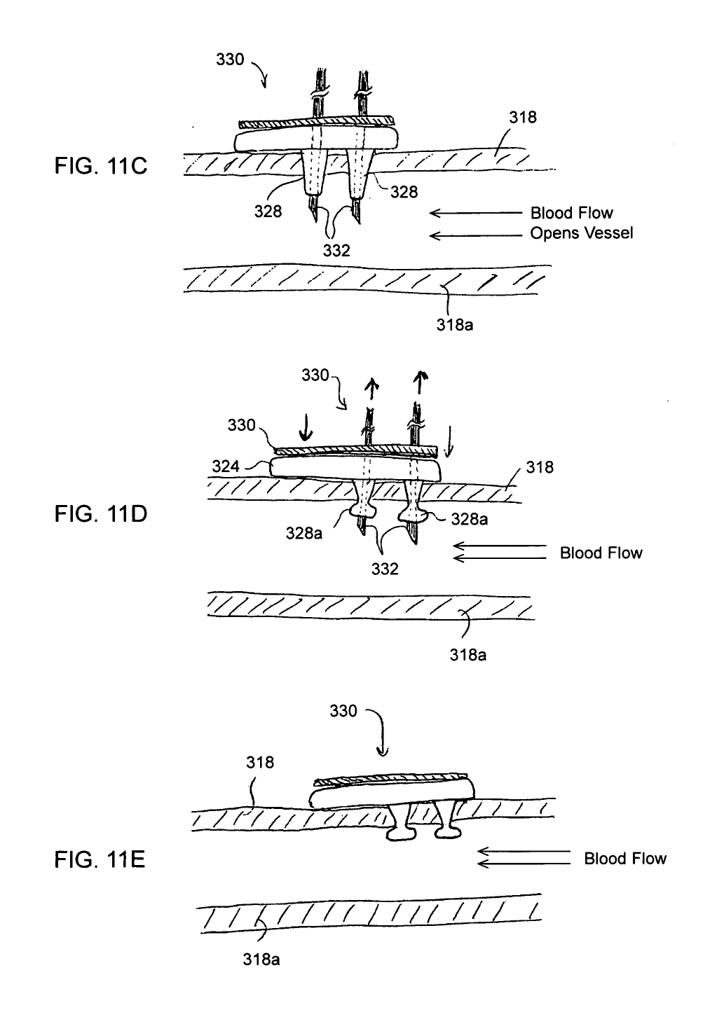

Another frame embodiment which does not necessarily require the use of a stopper mechanism for installation is that of FIGS. 6M-6M″. As shown in FIG. 11A, assembled frame 320 is delivered and positioned on and parallel to a surface of blood vessel 318. Advancement and downward pressure on frame 320 causes pins 332 and then sleeves 328 to penetrate into vessel wall 318 as well as the opposite vessel was. 318a (see FIG. 11B). Blood flow through the vessel is closed off causing the blood pressure to build up within the vessel. Once the downward force on frame 320 is released, the built up blood pressure forces open the vessel, causing pins 332 to retract from the opposite vessel wall 318a and normal blood flow is established (FIG. 11C). Next, while putting downward pressure on assembled frame 320, pins 332 are pulled proximally upward causing the distal portions 328a of sleeves 328 to buckle and sandwich vessel wall 318 between the distal portions and the underside of bottom plate 324 (FIG. 11D). Finally, pins 332 are completely withdrawn from the implant site leaving behind a securely implanted frame 330 (FIG. 11E).

Returning to the description of FIGS. 8A-8I, once frame 250 (or any of the above-described frames) is operatively positioned on the outer surface of vessel 2, a cutting instrument 270 is translated through shaft 264 and used to create an incision within the vessel wall (FIG. 8G). As described in detail above, the incision is made within the aperture/gap defined by the inner and outer frame members of frame 250. While not required, in certain embodiments, the frame is positioned on the vessel where the length of the aperture is positioned substantially perpendicular to the longitudinal axis of the blood vessel, thus producing an incision which is also substantially perpendicular to the axis of the blood vessel.

Cutting instrument 270 may have any suitable configuration, including a forward-facing distal blade member 272 having a shape and length substantially corresponding to and alignable with the frame aperture. As blade 272 is advanced, stopper 268 may be used to provide the necessary positive pressure against which the blade can be engaged in order to impale the tissue, thereby creating the incision. Alternatively, with frames having a crescent or arc shape, the blade member may be housed within the wall of shaft 264 and axially translatable through the aperture of a frame positioned on the distally facing edge of shaft 264. The blade member has a cross-sectional profile defining the shape and length of the incision to provide a tissue flap or jaw having the desired configuration, as discussed above. With an integrated arteriotomy system, such a configuration allows optimal use of space to minimize the profile of the components delivered within and through the tissue access site.

Alternatively, the cutting instrument may be equipped with a backward or proximally facing blade member (not shown), which is hinged or foldable to an axial or lower profile for easy entry into the vessel through the guidewire entry site. The blade member may be spring loaded or otherwise biased to a radially extended cutting position or actively expandable to such position, whereby it opens or is extended radially (e.g., in a 90° arc) when in the vessel. The proximally facing blade member is then pulled or advanced by stopper 268 proximally through the frame aperture to form the desired incision. Alternatively, the blade may be axially extendable whereby it opens or is extended 180° or so. The cutting instrument may then be used to slice or be drawn through the frame aperture to form the incision. Blades having a reducible profile or dimension may be hinged at an end of the blade or have one or two portions which are hinged at a more central location.

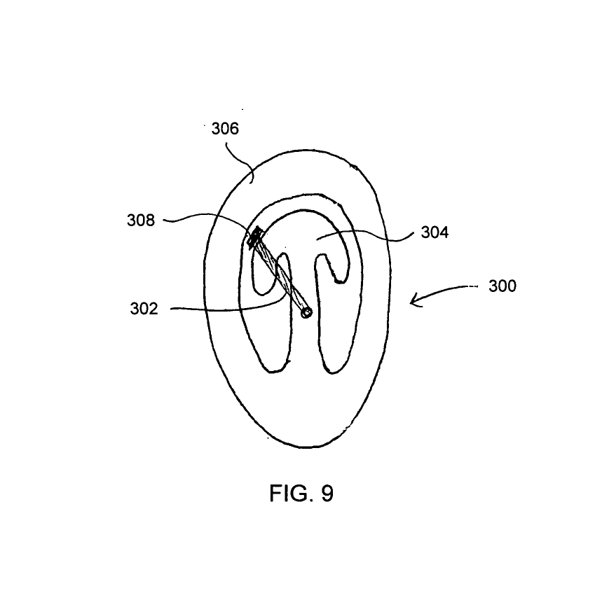

In still another embodiment, as illustrated in FIG. 9, the implantable device 300 may be equipped with a blade mechanism thereby eliminating the need for a separate cutting tool. For example, a blade arm 302 is rotatably coupled at a proximal end to an inner frame member 304 of device 300. Arm 302 has a length and arc of rotation such that a downwardly extending blade 308 accurately incises the tissue exposed in the gap between inner frame 304 and outer frame 306. Rotation of the blade arm may be performed with an instrument deliverable through or integrated with a shaft 264. Additionally, a stopper 268 may be used to provide the necessary positive pressure against which blade 308 can penetrate through the tissue wall and subsequently prevent injury to the inside back wall of the vessel.

Returning again to the description of the method illustrated in FIGS. 8A-8I, after incising vessel 2, cutting instrument 270 may be removed from the wound site with or without shaft 264. Next, as illustrated in FIG. 8H, instrumentation 274 for performing the endovascular procedure may be delivered over guidewire 262 and into vessel 2 through the opening 8 formed by separation of the inner and outer frame members 252, 254, i.e., depression of the tissue flat created by the frame to within the vessel lumen. When the endovascular procedure is complete and the associated instrumentation removed from vessel 2, the inner and outer frame members return to their co-planar or closed position, thereby aligning together or coapting the incision edges to seal the incision, as illustrated in FIG. 8I. The frame may be configured to also provide compressive force between the two opposing sides of the incised tissue so as to promote healing. Additionally or alternatively, other vessel closure means such as sutures, clips, staples, glue etc. may be used but are not necessary to create hemostasis as the implantable frames are intended to be self-closing.

While the method of FIGS. 8A-I has been described using a system of the present invention having integrated instrumentation, it is understood that this and other methods of the present invention may be performed without the use thereof, but rather, by use of standard surgical tools, including but not limited to scalpels and the like for making the arteriotomy incision as well as other instruments for performing one or more of the various other acts involved in the methods.

As evidenced in the above description, certain of the methods of the present are contemplated for using and implanting the devices of the present invention. The methods may comprise the act of providing a suitable device or system, etc. Such provision may be performed by the end user, i.e., the physician. In other words, the act of “providing” merely requires the end user obtain, access, approach, position, set-up, activate or otherwise act to provide the requisite object used in the subject method.

Yet another aspect of the invention includes kits having any combination of devices described herein—whether provided in packaged combination or assembled by a technician for operating use. A kit may include various shapes and sizes of the subject frames and/or components of a system for performing the subject methods. The subject kits may also include written instructions for implanting and using the subject devices. These instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the Internet, are provided. An example of this embodiment is a kit that includes a web address where the instructions can be viewed and/or from which the instructions can be downloaded. As with the instructions, this means for obtaining the instructions is recorded on suitable media.

As the totality of the above description reveals, the present invention overcomes many of the shortcomings of prior art vascular access and closure devices. The invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. Any number of the individual parts or subassemblies shown may be integrated in their design.

Where a range of values is provided, it is understood that every intervening value between the upper and lower limits of that range and any other stated or intervening value in that stated range is encompassed within the invention. Reference to a singular item, includes the possibility that there are plural of the same items present. More specifically, as used herein and in the appended claims, the singular forms “a, ” “an,” “said,” and “the” include plural referents unless the specifically stated otherwise. In other words, use of the articles allow for “at least one” of the subject item in the description above as well as the claims below.

Also, it is contemplated that any optional feature of the inventive variations described may be set forth and claimed independently, or in combination with any one or more of the features described herein. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth n the claims. Stated otherwise, unless specifically defined herein, all technical and scientific terms used herein are to be given as broad a commonly understood meaning as possible while maintaining claim validity.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A device for facilitating percutaneous access through a tissue surface, the device comprising:
a frame having a structure having first and second frame portions defining a space therebetween and having a tissue-engaging surface defining a plane,
wherein the first and second frame portions are biased to remain within the plane and wherein at least one of the frame portions is movable out of the plane.

2. The device of claim 1, wherein the frame structure is implantable.

3. The device of claim 2, wherein the frame structure is made of a bioresorbable material.

4. The device of claim 2, wherein the frame structure is made of a NITINOL.

5. The device of claim 1, wherein the frame structure further comprises at least one flexure point about which the frame portions are movable.

6. The device of claim 5, wherein a flexure point is formed by a cusp in the frame structure.

7. The device of claim 5, wherein the frame structure comprises at least two flexure points.

8. The device of claim 1, wherein the tissue-engaging surface is flat.

9. The device of claim 1, wherein the tissue-engaging surface is curved.

10. The device of claim 1, wherein the frame structure has a circular, elliptical, arc or crescent configuration.

11. The device of claim 1, wherein the first frame portion has a dimension which is larger than that of the second frame portion.

12. The device of claim 11, wherein the dimension is an arc length.

13. The device of claim 1, wherein the first frame portion is annular and the second frame portion resides within the first frame portion.

14. The device of claim 1, further comprising at least one barb attached to the frame structure.

15. The device of claim 1, wherein the frame structure is made of a rigid or semi-rigid material.

16. The device of claim 1, wherein the frame structure is made of a flexible or semi-flexible material.

17. The device of claim 16, wherein the frame structure is conformable to a tissue surface.

18. The device of claim 1, wherein the tissue-engaging surface is configured to lie substantially flush with the tissue surface.

19. A method for the controlled formation of an opening in a tissue surface, the method comprising:
positioning the device of claim 1 wherein the tissue-engaging surface engages the tissue surface; and
forming an incision within a portion of the tissue surface through the space defined within the frame structure.

20. The method of claim 19, wherein the opening is formed by moving at least one frame portion relative out of the plane defined by the tissue engaging surface.

21. The method of claim 20, wherein the space defines an arc shape and the incision forms at least one tissue flap within the tissue surface.

22. The method of claim 21, wherein moving at least one frame portion comprises pushing at least one tissue flap below the tissue surface.

23. The method of claim 20, wherein the tissue surface is a vessel wall and the opening is sized to sealingly accommodate the passage of instrumentation for use in an endovascular procedure.

24. The method of claim 19, further comprising securing the device to the tissue surface.

25. The method of claim 19 or 24, wherein one or more of the recited steps is performed with the provision of a positive pressure on the opposite side of the tissue surface.

26. A method for closing an opening in a tissue surface formed according to the method of claim 20, the method comprising:
allowing the frame portions to achieve their biased position wherein the incision edges are aligned with each other thereby creating hemostasis at the incision.

27. The method of claim 26 wherein the hemostasis is created solely by the frame structure.

28. The method of claim 26, wherein the hemostasis is created without the use of sutures.

29. A method for performing a percutaneous endovascular procedure, the method comprising:
positioning a frame defining an aperture on a vessel surface;
forming an incision having apposing edges within a portion of the vessel surface through the frame aperture;
moving one frame portion relative to the other frame portion whereby an opening is made within the vessel; and
translating at least one instrument through the opening to within the vessel to a location remote from the opening without dilating the opening.

30. The method of claim 29 wherein the opening is sized to sealingly engage the at least one instrument:

31. The method of claim 29 wherein the frame has a biased configuration, the method further comprising allowing the frame to return to the biased configuration by removing the at least one instrument from the opening;

32. The method of claim 31, wherein returning the frame to the biased configuration apposes the incision edges wherein hemostasis is provided at the incision.

33. The method of claim 29, wherein the incision is formed with a blade having a shape and length substantially similar to that of the aperture.

34. The method of claim 29, wherein at least one of positioning the frame and forming the incision comprises providing a positive pressure against an underside of the vessel surface.

35. A device for the controlled formation and closure of a vascular opening for the purposes of performing an endovascular procedure therethrough, the device comprising:
a configuration for forming an incision within a wall of a blood vessel, the incision having a predetermined shape and length; and
a means for biasing the incision in a closed position such that the vascular opening is biased closed.

36. The device of claim 35, wherein the configuration provides a crescent-shaped incision.

37. The device of claim 35, wherein the biasing means comprises at least one flexure point about which a first portion of the device is movable relative to a second portion of the device.

38. A method for the controlled formation and closure of a vascular opening for the purposes of performing an endovascular procedure therethrough, the method comprising:
placing a closure means on the blood vessel;
forming an incision having opposing edges within the blood vessel; and
biasing the incision edges to appose each other.

39. The method of claim 38, wherein the incision forms at least one tissue flap that is movable out of a plane defined by a surface of the blood vessel.

40. The method of claim 38, wherein the closure means defines an aperture through which the incision is made, wherein the device is placed on the blood vessel such that the incision formed is substantially perpendicular to the longitudinal axis of the blood vessel.

41. A method for performing an endovascular procedure, the method comprising:
placing a closure device on a blood vessel;
passing an endovascular tool past the closure device into the vessel; and
biasing the opening closed with the closure device.

42. The method of claim 41, wherein closure device remains permanently implanted.

43. The method of claim 42, wherein the closure device is made of a bioresorbable material.

44. The method of claim 41, wherein an incision is made in the blood vessel through which the endovascular tool is passed, and wherein the incision is made before the closure device is placed on the blood vessel.

45. The method of claim 41, wherein an incision is made in the blood vessel through which the endovascular tool is passed, and wherein the incision is made after the closure device is placed on the blood vessel.

46. A method for performing an endovascular procedure, the method comprising:
creating a controlled incision in a blood vessel;
inserting an endovascular tool through the incision;
attaching a closure device to the blood vessel tissue; and
biasing the incision into a closed state with the closure device.

47. The method of claim 46, wherein closure device remains permanently implanted.

48. The method of claim 47, wherein the closure device is made of a bioresorbable material.

49. The method of claim 46, wherein the incision is crescent-shaped.

50. The method of claim 46, wherein the incision forms at least one tissue flap that is movable out of a plane defined by a surface of the blood vessel.

51. The method of claim 46, wherein the closure device is attached to the blood vessel tissue before the controlled incision is created.

52. The method of claim 46, wherein the controlled incision is created before the closure device is attached to the blood vessel tissue.

53. A kit comprising a plurality of the devices described by claim 1, wherein the kit includes devices of different sizes.

54. A kit comprising a plurality of the devices described by claim 1, wherein the kit includes devices of different shapes.
